(19) United States
(12) Patent Application Publication
(10) Pub. No.: US 2024/0164948 A1
(43) Pub. Date: May 23, 2024

(54) A MULTILAYER COMPOSITE

(71) Applicant: National University of Singapore, Singapore (SG)

(72) Inventors: Barbaros Oezyilmaz, Singapore (SG); Gang Xu, Singapore (SG); Deqiang Zhang, Singapore (SG); Chee Tat Toh, Singapore (SG); Qian Yang, Singapore (SG); Yong Heng So, Singapore (SG)

(21) Appl. No.: 18/550,152

(22) PCT Filed: Mar. 14, 2022
(86) PCT No.: PCT/SG2022/050129

§ 371 Date: Sep. 12, 2023

(30) Foreign Application Priority Data
Mar. 12, 2021 (SG) 10202102579P

(51) Int. Cl. A61F 13/00; A61B 5/00; C23C 16/26; C23C 16/56

(52) U.S. Cl. CPC A61F 13/00063; A61B 5/6801; C23C 16/26; C23C 16/56

(57) ABSTRACT

There is provided a multilayer composite comprising at least one carbon layer having a plurality of cracks along a first directional axis, said cracks being spaced apart from each other in a periodic manner along a second directional axis, wherein said second directional axis is substantially perpendicular to said first directional axis in the same plane; and a ferroelectric polymer layer. There is also provided a method of producing a multilayer composite. There is further provided a bandage or biosensing device comprising the multilayer composite.

### REFERENCES TO RELATED APPLICATIONS

This application claims priority to Singapore application number 10202102579P filed on 12 Mar. 2021 with the Intellectual Property Office of Singapore, the disclosure of which is hereby incorporated by reference.

### TECHNICAL FIELD

The present invention generally relates to a multilayer composite comprising a carbon layer and a ferroelectric polymer layer. The present invention also relates to a method of producing the multilayer composite. The present invention further relates to a bandage comprising the multilayer composite.

### BACKGROUND ART

Graphene and other novel carbon derivatives are intensively investigated for its practical applications in the mechanical, optical, electronic, medical and chemical fields. While nano-sized graphene and its derivatives have been rigorously investigated in numerous aspects and with diverse applications, the study on large-area graphene sheet is markedly less intensive, due to the strict high requirements of CVD-based fabrication procedures. The state-of-art graphene-containing biosensors requires chemical etchants after CVD fabrication to transfer graphene from its growth substrate. Generally, graphene is transferred by a chemical etching method, inevitably containing contaminations and residues from etchant and intermediate supporting polymer film. The major bottlenecks for the wet transfer technique are the poor reproducibility and uniformity of the graphene. These challenges of producing large-area sheet graphene also which limits the industrial adoption and applications due to the disadvantages of wet chemical processes required in production.

A similar phenomenon is also observed in the aspect of biomedical applications of graphene where large-area graphene sheets are rarely reported. A state-of-art graphene-containing biosensor that is commercially available utilizes the conductive properties of graphene but does not have antibacterial properties. On the other hand, while there is a smart bandage for chronic wound management using a MXene-mediated porous graphene scaffold based on graphene oxide (GO) and reduced graphene oxide (rGO) with antibacterial properties, this bandage suffers from loss of conductivity and alternation of physicochemical properties, limiting it's smart applications in the biomedical environment.

The market of antimicrobial transparent bandage is remarkedly small, because biomaterials possessing these two properties simultaneously is extremely rare. To meet the antimicrobial requirements, silver was introduced into bandage/dressing to deliver an antimicrobial barrier which kills microorganisms within the dressing. However, the accumulation of silver nanoparticles in the human body is undesirable, due to the unknown healthcare issues of silver-containing bactericides. Alternatively, chlorhexidine gluconate (CHG) was integrated into a dressing adhesive to provide antimicrobial protection, where the growth of skin flora on the prepared skin was shown to be significantly inhibited for up to 7 days. However, as the modification and functionalization of CHG moieties on substrate surfaces is a tedious and multi-step procedure, this severely limits industrial-scale production.

Accordingly, there is a need for a material that overcomes, or at least ameliorates, one or more of the disadvantages mentioned above.

There is a need to provide a method of producing such a material that overcomes, or at least ameliorates, one or more of the disadvantages mentioned above.

There is a need to provide a bandage that overcomes, or at least ameliorates, one or more of the disadvantages mentioned above.

### SUMMARY

In one aspect, the present disclosure provides a multilayer composite comprising at least one carbon layer having a plurality of cracks along a first directional axis, said cracks being spaced apart from each other in a periodic manner along a second directional axis, wherein said second directional axis is substantially perpendicular to said first directional axis in the same plane; and a ferroelectric polymer layer.

Advantageously, the multilayer composite comprising the plurality of engineered periodic cracks may have a high coverage of carbon that can be regarded as a continuous phase of carbon. The engineered periodic cracks may allow for increased repeatability and consistency of producing a multilayer composite that is conductive in at least one directional axis. Thus, multilayered composites having such engineered periodic cracks are advantageous over, for example, graphene layers containing randomly generated cracks, where there is no control over how the cracks are generated, leading to regions or islands of graphene which do not form a continuous phase with the other regions or islands of graphene, such that the conductivity across such randomly generated cracks is nil.

Further advantageously, the multilayer composite may demonstrate low amounts of chemical contaminant, thus improving its purity and suitability of usage in a wide array of applications.

Still further advantageously, the multilayer composite may contain relatively large area graphene sheets compared to conventional graphene which are typically nanosized or microsized, leading to ease in scaling up and capable of industrial-scale manufacturing in the mechanical, optical, electronic, and chemical fields.

Still further advantageously, the multilayer composite may exhibit good conductivity with a sheet resistance below 200 Ω/sq, thus enabling electrical and electronic applications with the composite.

Still further advantageously, the multilayer composite may exhibit good transparency with optical transmission above 95% in the normal light spectrum, thus enabling its use in applications where visibility of surfaces covered by the multilayer composite is important.

Still further advantageously, the multilayer composite may exhibit antimicrobial activity, thus enabling its use as an antimicrobial material.

Still further advantageously, the multilayer composite may reduce bacterial or viral infection rates and accelerate wound healing when used as a medical application.

Still further advantageously, the multilayer composite may promote wound healing via control and promotion of human mesenchymal stem cells' osteogenic differentiation, enhancing cell attachment and blood vessel formation, simulating stem cell immigration and proliferation, thus providing benefits as a biomedical material.

Still further advantageously, the multilayer composite may be functionalized with a biomaterial, thus providing benefits as a biomedical material.

In another aspect, the present disclosure provides a method of producing a multilayer composite, comprising the steps of:
(a) providing at least one carbon layer on a growth substrate;
(b) applying a ferroelectric polymer layer on said carbon layer;
(c) polarizing said ferroelectric polymer layer; and
(d) forming a plurality of cracks in said carbon layer along a first directional axis, said cracks being spaced apart from each other in a periodic manner along a second directional axis, wherein said second directional axis is substantially perpendicular to said first directional axis in the same plane of said carbon layer.

Advantageously, the method of producing a multilayer composite may introduce periodic cracks into the carbon layer of the multilayer composite to tailor permeability and breathability of the multilayer composite, thus enhancing the effectiveness of the multilayer composite when used, for example, as a medical bandage. The method of producing a multilayer composite with engineered periodic cracks may further advantageously allow for increased repeatability and consistency of producing a multilayer composite that is conductive in at least one directional axis.

Further advantageously, the method of producing a multilayer composite may provide a dry-phase production technique, thus eliminating the use of, and potential resultant contamination of the composite with chemical etchants.

Still further advantageously, the method of producing a multilayer composite with a dry-phase production technique allows for the potential of patterning on the composite as well as facile transfer onto a much wider array of suitable target substrates as compared to using chemical etchants.

Still further advantageously, the method of producing a multilayer composite may provide a facile production technique to produce large-area carbon laminae such as large area graphene sheets, thus improving industrial scalability of graphene materials.

Still further advantageously, the method of producing a multilayer composite may produce a large-area carbon layer such as a large-area graphene sheet, thus improving industrial scalability of graphene materials.

Still further advantageously, the method of producing a multilayer composite may consist of repeatable procedures to obtain different orders, thickness, and morphologies of the composite, thus improving the mechanical and physical properties of the multilayer composite.

In yet another aspect, the present disclosure provides a bandage comprising a multilayer composite, the multilayer composite comprises at least one carbon layer having a plurality of cracks along a first directional axis, said cracks being spaced apart from each other in a periodic manner along a second directional axis, wherein said second directional axis is substantially perpendicular to said first directional axis in the same plane; and a ferroelectric polymer layer.

Due to the engineered periodic cracks in the carbon layer, in addition to the advantages mentioned above, the engineered periodic cracks may allow for increased control of gas permeation and breathability in the bandage. Advantageously, the bandage comprising the multilayer composite may be capable of electronic stimulation for wound healing due to the conductivity of the multilayer composite, thus improving benefits for skin wound healing by promoting cell migration and proliferation.

Further advantageously, the bandage comprising the multilayer composite may be capable of promoting wound healing due to the antimicrobial properties of the multilayer composite, thus providing a medical benefit over conventional medical bandages.

Still further advantageously, the bandage comprising the multilayer composite may be capable of visual monitoring of wounds due to the transparency of the multilayer composite, thus providing a medical benefit over conventional medical bandages.

Still further advantageously, the bandage comprising the multilayer composite contains a hydrophobic surface with a contact angle higher than 90 degrees, thus may aid in reducing blood absorption and induction of secondary bleeding by the bandage and promoting ease of bandage removal, as well as being able to reduce blood loss by acting as an impervious layer, as well as being able to promote blood clotting and coagulation by preventing seepage of blood through the bandage, thus providing a medical benefit over conventional medical bandages.

In yet another aspect, the present disclosure provides a biosensing device comprising a multilayer composite, the multilayer composite comprises at least a carbon layer having a plurality of cracks along a first directional axis, said cracks being spaced apart from each other in a periodic manner along a second directional axis, wherein said second directional axis is substantially perpendicular to said first directional axis in the same plane; and a ferroelectric polymer layer.

Advantageously, the biosensing device comprising the multilayer composite may be capable of functioning as a smart biosensor due to the conductivity and strength of the multilayer composite, thus providing benefits such as sensing biodata and wound diagnostics of parameters such as pus discharge, wound pH and temperature with improved sensitivity and lower limits of detection than conventional biosensors.

Further advantageously, the bandage comprising the multilayer composite may be capable of rapid virus sensing due to the ability to detect antigens, characteristic proteins, DNA, and RNA, thus providing a benefit over conventional medical bandages.

### Definitions

The following words and terms used herein shall have the meaning indicated:

The term “multilayer” when used in conjunction with a composite or material is to be interpreted broadly to refer to the composite or material as having a layered structure, where the layered structure is made up of two or more layers. The term “bilayer” then refers to having two layers.

The term ‘composite’ is to be interpreted broadly to refer to a product or item that is made up of multiple separate and distinct materials.

The term ‘graphene’ is to be interpreted broadly to refer to a single or multilayer two-dimensional planar sheet of carbon, wherein each carbon atom is covalently bonded to 3 other carbon atoms via a σ-bonds in sp2 orbits.

The term ‘amorphous carbon’ is to be interpreted broadly to refer to a single or multilayer two-dimensional or three-dimensional amorphous sheet of carbon without any defined crystallinity, wherein the carbon atoms may be covalently bonded to other carbon atoms via σ-bonds in sp, sp2 or sp3 orbits, or combinations thereof.

The term ‘ferroelectric polymer’ is to be interpreted broadly to refer to any polymer that can be engineered with the use of an external electric field to exhibit ferroelectric properties, such that the polymer will exhibit a permanent electric polarization charge on the surface that can be switched or reversed when subjected to yet another external electric field of a different polarity or direction.

The term ‘growth substrate’ is to be interpreted broadly as the substrate that carbon atoms are grown on to form layers of carbon.

The term ‘target substrate’ is to be interpreted broadly as the secondary substrate to which the delaminated multilayer composite is to be transferred onto.

The term ‘delaminate’ is to be interpreted broadly to refer to the separation or the act of separating individual or multiple layers of constituents from a composite stack of different layered constituents.

The term ‘bandage’ is to be interpreted broadly to refer to a strip or sheet of material that is applied onto any bodily surface either on its own, or held in place with the support of a contraption such as adhesive tapes, medical dressing or stitches.

The term ‘one-dimensional’ is to be interpreted broadly to refer to a geometric description of a structure with only one main significant geometrical dimension of interest such as length, and two other insignificant geometrical dimensions such as width and height, in a three-dimensional geometric space.

The term ‘two-dimensional’ is to be interpreted broadly to refer to a geometric description of a structure with only two main significant geometrical dimensions of interest examples being (a) length and width, or (b) length and height, or (c) width and height, and one remaining insignificant geometrical dimension such as (a) height, (b) width and (c) height, in a three-dimensional geometric space.

The term ‘planar’ is to be interpreted broadly to refer to a geometric description of a two-dimensional flat structure with only two main significant geometrical dimensions of interest being the length and width, and one remaining insignificant geometrical dimension being the height or thickness. In a cartesian coordinate system, this flat surface would be represented by the x-axis and y-axis, with a non-existent or insignificant z-axis.

The term ‘crack’ is to be interpreted broadly to refer to a line or void on a planar surface that has split apart.

The term ‘crack length’ is to be interpreted broadly to refer to the length of the crack on a planar two-dimensional surface. The length is the most significant geometric dimension of the crack, but its direction is not limited to any particular axis on a cartesian coordinate system, and its directional vector may vary at different points of the crack. Although the direction of the crack may vary, a straight line through both end-points of the crack can be regarded as its directional vector, and being along (or extending along) the first directional axis in the plane of the carbon layer. In this manner, the crack length may be regarded as being parallel to the first directional axis.

The term ‘crack width’ is to be interpreted broadly to refer to the width of the crack on a planar two-dimensional surface. The width is the secondary geometric dimension of the crack after the length of the crack, and its direction is substantially perpendicular to the directional vector of the crack length at any point of the crack.

The term ‘crack gap width’ is to be interpreted broadly as the distance between widths of two discreet, neighbouring cracks, where the distance to be measured is at the points where the distance between two discreet, neighbouring cracks is at its largest. The ‘crack gap width’ is then regarded as the spacing apart of the cracks from each other in the periodic manner along the second directional axis.

The word “substantially” does not exclude “completely” e.g., a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range.

For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

Exemplary, non-limiting embodiments of a multilayer composite will now be disclosed.

The multilayer composite comprises at least one carbon layer having a plurality of cracks along a first directional axis, said cracks being spaced apart from each other in a periodic manner along a second directional axis, wherein said second directional axis is substantially perpendicular to said first directional axis in the same plane; and a ferroelectric polymer layer.

The carbon layer may be a two-dimensional carbon layer. The carbon layer may be a planar layer. The carbon layer may be a two-dimensional planar layer.

The carbon of the carbon layer may be selected from the group consisting of a carbon layer, an amorphous carbon, a graphene, a graphene oxide, a reduced graphene oxide, a graphite layer or a combination thereof. The amorphous carbon may be monolayer amorphous carbon.

The carbon layer may have a thickness in the range of about 0.34 nm to about 100 nm, about 0.34 nm to about 1 nm, about 0.34 nm to about 5 nm, about 0.34 nm to about 10 nm, about 0.34 nm to about 20 nm, about 0.34 nm to about 50 nm, about 1 nm to about 100 nm, about 5 nm to about 100 nm, about 10 nm to about 100 nm, or about 20 nm to about 100 nm.

The carbon layer may have an area in the range of about 0.1 cm² to about 600 cm², about 0.1 cm² to about 600 cm², about 10 cm² to about 600 cm², about 50 cm² to about 600 cm², about 100 cm² to about 600 cm², about 300 cm² to about 600 cm², about 0.1 cm² to about 10 cm², about 0.1 cm² to about 50 cm², about 0.1 cm² to about 100 cm², or about 0.1 cm² to about 300 cm².

The cracks may be one-dimensional cracks. The cracks may be regarded as periodic cracks.

The cracks may be two-dimensional cracks. A two-dimensional crack may have substantially similar crack length and crack widths. Example of a two-dimensional crack may be a crack resembling a spherical, elliptical, or rhombic shape.

The plurality of cracks may be a combination of a plurality of one-dimensional cracks and a plurality of two-dimensional cracks.

The cracks along the first dimensional axis may be regarded as extending along the first dimensional axis whereby the crack length of the cracks is parallel to the first dimensional axis. Where the crack is not a perfect straight line or a perfect two-dimensional shape, the crack length is to be regarded as a straight line that connects two end-points of the crack (be it the one-dimensional crack or the two-dimensional crack, where the two end-points are regarded as end-points that are furthest from each other) and this straight line is then substantially parallel to the first directional axis.

The cracks in the carbon layer may have a crack length in the range of about 100 nm to about 100 μm, about 1 μm to about 100 μm, about 10 μm to about 100 μm, about 50 μm to about 100 μm, about 100 nm to about 1 μm, about 100 nm to about 10 μm, or about 100 nm to about 50 μm.

The cracks in the carbon layer may have a crack width in the range of about 100 nm to about 10000 nm, about 200 nm to about 10000 nm, about 500 nm to about 10000 nm, about 1000 nm to about 10000 nm, about 2000 nm to about 10000 nm, about 5000 nm to about 10000 nm, about 100 nm to about 200 nm, about 100 nm to about 500 nm, about 100 nm to about 1000 nm, about 100 nm to about 2000 nm, or about 100 nm to about 5000 nm.

The cracks in the carbon layer may have a crack gap width spaced apart along the second directional axis, where the crack gap width is in the range of about 100 nm to about 100 μm, about 1 μm to about 100 μm, about 10 μm to about 100 μm, about 50 μm to about 100 μm, about 100 nm to about 1 μm, about 100 nm to about 10 μm, or about 100 nm to about 50 μm.

The carbon layer may be considered as a continuous phase, which is conductive in at both the first directional axis of the cracks, as well as the second directional axis that is substantially perpendicular to the first directional axis, despite the presence of periodic cracks on the carbon layer.

The carbon layer may be considered as a continuous phase, which is conductive in at least the first directional axis of the cracks, despite the presence of periodic cracks on the carbon layer.

The carbon layer may have a carbon coverage in the range of about 80% to about 99%, about 90% to about 99%, about 95% to about 99%, about 80% to about 85%, about 80% to about 90%, or about 80% to about 95%.

The carbon layer may have an optical transmission of natural light in the range of about 95% to about 99%, about 96% to about 99%, about 97% to about 99%, about 98% to about 99%, about 95% to about 96%, about 95% to about 97%, or about 95% to 98%.

The carbon of the carbon layer may be attached with a biomaterial. The biomaterial may be bonded with the carbon. The carbon may be modified to enable attachment or bonding with the biomaterial. The carbon may be functionalised with the biomaterial. The biomaterial may be a therapeutic agent or a detection agent. The therapeutic agent may be an agent capable of exerting a therapeutic effect on a subject when the multilayer composite is placed on a body surface of the subject. The therapeutic agent may be able to stimulate inflammation, angiogenesis, wound contraction, and remodelling for accelerative wound healing, such as for chronic wounds, bedsore wounds, skin cutting wounds, burn wounds, venous ulcers, arterial ulcers or diabetic (neuropathic foot ulcers).

The therapeutic agent may be an anti-cancer drug, an anti-clotting agent, a wound healing agent, an anti-inflammation agent, an antioxidant, a vitamin or a pain reducing agent. The therapeutic agent is not particularly limited and an exemplary therapeutic agent may include diclofenac, salicylic acid, sulphamethoxypyridazine, phenoxymethylpenicillin, phenol red, valproate, bezafibrate, furosemide, indomethacin, mefenamic acid, piroxicam, tolbutamide, warfarin, cystine, sodium cromoglycate, tetrachlorodecaoxide, hydrogen peroxide, carbamide peroxide, ferric subsulfate, sodium perborate, potassium nitrate, CHS-828, OXI-4503, PX-12, CPI-613, double stranded DNA, single stranded DNA, double stranded RNA, single stranded RNA, messenger RNA or combinations thereof.

The detection agent may act as a probe for a target analyte and be capable of detecting the target analyte, such as when the multilayer composite is placed on a body surface of a mammal; or when made into or as part of a detection device. The detection agent may be an antibody, a protein, a nucleic acid or a combination thereof. The antibody or protein is not particularly limited and an exemplary antibody or protein may include an TgG antibody, an IgA antibody, an IgM antibody, an IgE antibody, an IgD antibody, a SAR-COV 2 antibody, a MERS-COV antibody, a Zika virus antibody, a HIV antibody, a Polio antibody, a Tenanus antibody, an Influenza antibody, an antinuclear antibody, an anti-transglutaminase antibody, an anti-ganglioside antibody, an anti-actin antibody, an anti-thyroid antibody, a spike glycoprotein, a spike protein, a membrane protein, an envelope protein polysaccharide, a peptides, an antigen or a combination thereof.

The carbon of the carbon layer may be attached with a non-organic material. The non-organic material may be bonded with the carbon. The carbon may be modified to enable attachment or bonding with the non-organic material. The non-organic material may be a nanometal, a nano composite or a nanoalloy.

The nanometal may be a metal that exhibits antibacterial, antiviral, or antifungal properties. The nanometal is not particularly limited and exemplary nanometals may include titanium, cobalt, nickel, copper, zinc, zirconium, molybdenum, tin, lead, silver, or combinations thereof.

The nano composite may be a composite comprising one or more nanometals that exhibits antibacterial, antiviral, or antifungal properties in a matrix. The nanometal is not particularly limited and exemplary nanometals may include titanium, cobalt, nickel, copper, zinc, zirconium, molybdenum, tin, lead, silver, or combinations thereof. The composite matrix may be a metal, a wood, a wood derivative, a polymer, a monomer, a plastic, a ceramic or combinations thereof.

The nanoalloy may be an alloy comprising at least one metal component that exhibits antibacterial, antiviral, or antifungal properties. The nanoalloy components are not particularly limited and exemplary nanometals may include titanium, cobalt, nickel, copper, zinc, zirconium, molybdenum, tin, lead, iron, platinum, silver, aluminium, palladium, gold or combinations thereof.

The ferroelectric polymer layer may be a two-dimensional ferroelectric polymer layer. The ferroelectric polymer layer may be a planar layer. The ferroelectric polymer layer may be a two-dimensional planar layer.

The ferroelectric polymer of the ferroelectric polymer layer may be selected from the group consisting of a fluoropolymer, a polyamide or combinations thereof.

The fluororopolymer of the ferroelectric polymer layer may be selected from the group consisting of polyvinylidene fluoride (PVDF), polyvinylfluoride (PVF), polytetrafluoroethylene (PTFE), polychlorotrifluoroethylene (PCTFE), fluorinated ethylene-propylene (FEP), trifluoroethylene (TrFE), polyethylenetetrafluoroethylene (ETFE), polyethylenechlorotrifluoroethylene (ETCFE), perfluoroalkoxy polymer (PFAP), perfluoropolyether (PFPE) and a combination thereof. The fluoropolymer may be poly[(vinylidenefluoride-co-trifluoroethylene] (PVDF-TrFE).

The polyamide of the ferroelectric polymer layer may be selected from the group consisting of polyamide 5 (Nylon 5), polyamide 11 (Nylon 11), polyamide 12 (Nylon 12), polyamide 66 (Nylon 66), polyamide 610 (Nylon 610), polyamide 66/610 (Nylon 66/610), polyamide 6/12 (Nylon 6/12), polyamide 666 (Nylon 666 or 6/66), polyamide 6/69 (Nylon 6/6.9), Nylon 1010, Nylon 1012, odd nylons, even nylons, odd-odd nylons, amorphous polyamides, Nylon PACM-12, polyacrylamide, aromatic polyamide, poly-paraphenylene terephthalamide, Nomex, p-phenylene terephthalamide, polyphthalamide and a combination thereof.

The vinyl polymer of the ferroelectric polymer layer may be selected from the group consisting of polyethylene, polystyrene, polyvinyl chloride, polyvinyl acetate, polyacrylonitrile, polyvinylidene, polyvinylidene fluoride, poly(vinylidene fluoride-trifluoroethylene), poly(vinylidene fluoride), vinylidene fluoride and a combination thereof.

The ferroelectric polymer layer may have a thickness in the range of about 300 nm to about 2000 nm, about 500 nm to about 2000 nm, 800 nm to about 2000 nm, about 1000 nm to about 2000 nm, about 1500 nm to about 2000 nm, about 300 nm to about 500 nm, about 300 nm to about 800 nm, about 300 nm to about 1000 nm, about 300 nm to about 1500 nm, or about 500 nm to about 1500 nm.

The ferroelectric polymer layer may have an area in the range of about 0.1 cm² to about 600 cm², about 0.1 cm² to about 600 cm², about 10 cm² to about 600 cm², about 50 cm² to about 600 cm², about 100 cm² to about 600 cm², about 300 cm² to about 600 cm², about 0.1 cm² to about 10 cm², about 0.1 cm² to about 50 cm², about 0.1 cm² to about 100 cm², or about 0.1 cm² to about 300 cm².

The ferroelectric polymer layer may have an optical transmission of natural light in the range of about 95% to about 99%, about 96% to about 99%, about 97% to about 99%, about 98% to about 99%, about 95% to about 96%, about 95% to about 97%, or about 95% to 98%.

The multilayer composite may comprise a plurality of bilayers, where each bilayer consists of one carbon layer and one ferroelectric polymer layer. Alternatively, the multilayer composite may comprise a plurality of stacked layers, where each stacked layer consists of more than one carbon layers and one ferroelectric polymer layer. In the stacked layer, there may be two to five carbon layers per one ferroelectric polymer layer. In the stacked layer, the pattern of laying the (A) carbon layer and (B) ferroelectric polymer layer may be in any particular permutation of a set number of layers as long as at least one of each layer is included, for example in a 5 layer multilayer composite: AAAAB, AAABB, AAABA, AABBB, AABBA, AABAA, AABAB, ABBBB, ABBBA, ABBAB, ABABB, ABABA, ABABB, ABBAA, ABAAA, ABAAB, BAAAA, BAAAB, BAABB, BABBB, BAABB, BABAB, BBAAB. BAABA. BBAAA. BABAA, BBABA. BBBAA. BBBBA. BBBAB, BABBB, BBABB.

The multilayer composite may comprise a mix of a bilayer and a stacked layer, or a mix of a plurality of bilayers and a stacked layer, or a mix of a bilayer with a plurality of stacked layer, or a mix of a plurality of bilayers and a plurality of stacked layers.

The number of bilayers or stacked layers may range from 1 to 5. The multilayer composite maybe conductive. The multilayer composite may have a surface charge due to an electric polarization technique. The surface charge may be positive or negative, or positive and negative on different surfaces of the multilayer composite.

Where the multilayer composite has a positive charge, the positively charged multilayer composite may be able to inhibit bacterial growth and adhesion by disrupting the bacterial cell membranes via electrostatic interaction.

The surface charge may be in the range of about 1 μC/cm² to about 100 μC/cm², about 5 μC/cm² to about 100 μC/cm², about 10 μC/cm² to about 100 μC/cm², about 30 μC/cm² to about 100 μC/cm², about 50 μC/cm² to about 100 μC/cm², about 1 μC/cm² to about 5 μC/cm², about 1 μC/cm² to about 30 μC/cm², about 1 μC/cm² to about 50 μC/cm², 1 μC/cm² to about 20 μC/cm², or about 1 μC/cm² to about 15 μC/cm².

The multilayer composite may have a sheet resistance in the range of about 100 Ω/sq to about 200 Ω/sq, about 150 Ω/sq to about 200 Ω/sq, or about 100 Ω/sq to about 150 Ω/sq. The sheet resistance is measured between each bilayer or between each stacked layer.

The multilayer composite may have an improved optical transmission of natural light as compared to the optical transmission of the carbon layer or the ferroelectric polymer layer individually, wherein the improvement in the optical transmission is about 0.1% to about 5%, about 0.5% to about 5%, about 1% to about 5%, about 2% to about 5%, about 3% to about 5%, about 4% to about 5%, about 0.1% to about 0.5%, about 0.1% to about 1%, about 0.1% to about 2%, about 0.1% to about 3%, or about 0.1% to about 4%.

The multilayer composite may exhibit antibacterial properties. The multilayer composite may exhibit antibacterial properties towards gram-positive bacteria, such as S. epidermis, S. aureus, S. pyogenes, S. saprophyticus, E. faecalis, S. pneumoniae or combinations thereof.

The multilayer composite may exhibit antibacterial properties. The multilayer composite may exhibit antibacterial properties towards gram-negative bacteria, such as E. coli, Salmonella, Shigella, Enterobacteriaceae, P. aeruginosa, C. trachomatis, Y. pestis, P. mirabilis, E. cloacae, S. marcescens or combinations thereof.

In the multilayer composite, the ferroelectric polymer layer may be placed above the carbon layer.

In the multilayer composite, the carbon layer and the ferroelectric polymer layer may be in physical contact with each other.

In the multilayer composite, the carbon layer and the ferroelectric polymer layer may be held in place together by intermolecular force, hydrogen bonding, ion-induced dipole forces, ion-dipole forces, van der Waal forces, gravitational force, ionic bonds, covalent bonds, or combinations thereof.

The multilayer composite may be attached or connected to an external electrical source supplying pulse of electricity, with the source voltage in the range of about 1 to about 1000 volts, about 10 to about 1000 volts, about 50 to about 1000 volts, about 100 to about 1000 volts, about 200 to about 1000 volts, about 300 to about 1000 volts, about 400 to about 1000 volts, about 500 to about 1000 volts, about 10 to about 500 volts, about 50 to about 500 volts, about 100 to about 500 volts, or about 250 to about 500 volts.

The external electrical source may have source pulse rate in the range of about 1 to about 1000 Hz, about 10 to about 1000 Hz, about 50 to about 1000 Hz, about 100 to about 1000 Hz, about 200 to about 1000 Hz, about 300 to about 1000 Hz, about 400 to about 1000 Hz, about 500 to about 1000 Hz, about 10 to about 500 Hz, about 50 to about 500 Hz, about 100 to about 500 Hz, or about 250 to about 500 Hz.

Exemplary, non-limiting embodiments of a method of producing a multilayer composite will now be disclosed.

The method of producing a multilayer composite comprises the steps of:
(a) providing at least one carbon layer on a growth substrate;
(b) applying a ferroelectric polymer layer on said carbon layer;
(c) polarizing said ferroelectric polymer layer; and
(d) forming a plurality of cracks in said carbon layer along a first directional axis, said cracks being spaced apart from each other in a periodic manner along a second directional axis, wherein said second directional axis is substantially perpendicular to said first directional axis in the same plane of said carbon layer.

The growth substrate is not particularly limited and exemplary materials may be copper films, copper plates, copper articles, copper alloys, nickel films, nickel plates, nickel articles, nickel alloys, platinum films, platinum plates, platinum articles, platinum alloys, cobalt films, cobalt plates, cobalt articles, cobalt alloys, germanium films, germanium plates, germanium articles, germanium alloys, sapphire, sapphire articles, silicon carbide, silicon carbide articles, silicon oxide, silicon oxide films, silicon oxide plates or silicon oxide articles.

The at least one carbon layer may be provided on the growth substrate in step (a) by chemical vapor deposition (CVD), physical vapor deposition (PVD), pulsed laser deposition (PLD) or epitaxial growth from high temperature annealing of carbon containing materials.

The ferroelectric polymer layer may be applied onto the carbon layer in step (b) by a number of coating processes such as spin-coating, Langmuir Blodgett method, dip coating, slot die, bar coating, doctor blade or wire coating.

The method may comprise the step of, after the applying step (b), the step of (b1) annealing the ferroelectric polymer on the carbon layer.

The annealing step (b1) may be carried out at a temperature from about 50° C. to about 200° C., about 80° C. to about 200° C., about 100° C. to about 200° C., about 120° C. to about 200° C., about 150° C. to about 200° C., about 180° C. to about 200° C., about 50° C. to about 80° C., about 50° C. to about 100° C., about 50° C. to about 120° C., about 50° C. to about 150° C., about 50° C. to about 180° C., or about 100° C. to about 150° C.

The annealing step (b1) may be carried out at a duration from about 1 minute to about 24 hours, about 1 hour to about 24 hours, about 6 hours to about 24 hours, about 12 hours to about 24 hours, about 18 hours to about 24 hours, about 1 minute to about 1 hour, about 1 minute to about 6 hours, about 1 minute to about 12 hours, about 1 minute to about 18 hours, or about 1 hour to about 12 hours.

The ferroelectric polymer layer may be applied onto the carbon layer in step (b) by using a roller, a heated roller or a press roller.

The ferroelectric polymer layer may be polarised in step (c) by introducing an external electric field with opposite polarities across both surfaces of said ferroelectric polymer layer, said surfaces being opposite to each other. The polarising step may be carried out by corona poling, plasma ionization, linear polarization, circular polarization, and elliptical polarization.

During the polarising step (c), the temperature of the ferroelectric polymer layer may be raised to about 70° C. to about 100° C., about 80° C. to about 100° C., about 90° C. to about 100° C., about 70° C. to about 80° C., or about 70° C. to about 90° C.

The forming step (d) may be a step of (d1) applying a pressure in excess of about 0.5 N/cm² or at least about 0.5 N/cm² on the ferroelectric polymer. The pressure may be in the range of about 0.5 N/cm² to about 10 N/cm². The pressure may be in excess of or at least about 0.5 N/cm², 1 N/cm², about 2/N/cm², about 3/N/cm², about 4/N/cm², about 5/N/cm², about 6/N/cm², about 7/N/cm², about 8/N/cm², or about 9/N/cm², until about 10/N/cm².

The forming step (d) may be a step of (d2) removing the carbon/ferroelectric polymer layers from the growth substrate at an increased peeling speed. The removing step (d2) may be before the applying step (d1) or may be after the applying step (d1) or may be during the applying step (d1). The plurality of cracks may be formed in the applying step (d1), the removing step (d2) or in both the applying step (d1) and the removing step (d2).

The removing step (d2) may induce periodic line cracks in the carbon layer. When the peeling speed is increased, the periodic line cracks induced are longer in crack length and higher in crack density as compared to when the peeling speed is lower.

The removing step (d2) may also amplify existing periodic line cracks in the carbon layer. When the peeling speed is increased, the existing periodic line cracks become amplified in crack length and in crack density as compared to when the peeling speed is lower.

The method may further comprise, before the applying step (d), the steps of: (d3) applying a release adhesive on the ferroelectric polymer layer; and (d4) optionally, removing the carbon/ferroelectric polymer layers from the growth substrate.

The removing step (d2) or (d4) may be carried out using a roller, a heated roller, a press roller, or a combination thereof.

The removing step (d2) or (d4) may be regarded as roller-assisted mechanical peeling.

The removing step (d2) may be performed at a peeling speed in excess of or at least about 101 mm/s, about 150 mm/s, about 200 mm/s, about 300 mm/s, or about 500 mm/s, until about 1000 mm/s, or until the working tolerance of the peeling device (such as the roller as mentioned above).

The removing step (d4) may be performed at a peeling speed at or below about 100 mm/s, about 80 mm/s, about 50 mm/s, about 30 mm/s, or about 10 mm/s.

Where the removing step (d4) is not present, the forming step (d) may be the removing step (d2) only or both the removing step (d2) and the applying step (d1) in any sequence. Where the removing step (d4) is present, the forming step (d) may comprise the applying step (d1) only. The removing step (d4) may be carried out at a peeling speed that does not form the cracks as this is covered by removing step (d2).

The method may further comprise, after the applying step (d) the step of: (d5) removing the release adhesive from the ferroelectric polymer layer.

The applying step (d) or the applying step (d5) may be undertaken at a temperature in the range of about 30° C. to about 160° C., about 50° C. to about 160° C., about 80° C. to about 160° C., about 100° C. to about 160° C., about 120° C. to about 160° C., about 30° C. to about 50° C., about 30° C. to about 80° C., about 30° C. to about 100° C., about 30° C. to about 120° C., or about 100° C. to about 140° C.

The release adhesive in step (d3) and (d5) is not particularly limited and an exemplary tape may be a thermal release tape, an epoxy release tape, a stretch release tape or an electric release tape.

The removal step (d5) may be undertaken by mechanical peeling, optionally assisted by the application of heat, electricity, tensile force, or chemical release agents.

The method may be used to form a multilayer composite comprising more than one carbon layers and one ferroelectric polymer layer. Here, the providing step (a) may comprise the steps of:
(a1) applying a first carbon layer on said growth substrate;
(a2) applying a second carbon layer on said first carbon layer; and
(a3) repeating said applying step (a2) for a number of times, such as one to three times.

The method may be used to produce a plurality of multilayer composites, each multilayer composite comprising a bilayer consisting of one carbon layer and one ferroelectric polymer layer. Alternatively, the method may be used to produce a plurality of multilayer composites, each multilayer composite comprising a stacked layer consisting of more than one carbon layers and one ferroelectric polymer layer. In the stacked layer, there may be two to five carbon layers per one ferroelectric polymer layer.

In order to produce the plurality of multilayer composites, the method may further comprise the steps of:
(e) repeating steps (a) to (d) to form a subsequent multilayer composite; and
(f) laminating said subsequent multilayer composite onto a multilayer composite previously produced by steps (a) to (d) or steps (a) to (f).

Steps (e) and (f) may be repeated as desired to form the plurality of multilayer composites and the terms “subsequent multilayer composite” and “multilayer composite previously produced” are then to be regarded as the successive (or next) multilayer composite and the previously produced multilayer composite, respectively. Therefore, where steps (e) and (f) are repeated, this will result in laminating a successive multilayer composite onto a previously produced multilayer composite.

The plurality of multilayer composites may comprise a mix of a multilayer composite having a bilayer and a multilayer composite having a stacked layer, or a mix of a plurality of multilayer composites having bilayers and a multilayer composite having a stacked layer, or a mix of a multilayer composite having a bilayer with a plurality of multilayer composites having stacked layer, or a mix of a plurality of multilayer composites having bilayers and a plurality of multilayer composites having stacked layers.

The multilayer composite may be applied or transferred onto a target substrate during step (d) or after steps (d5) and (f). The target substrate is not particularly limited and exemplary materials may be a metal, a wood, a wood derivative, a polymer, a monomer, a plastic, a ceramic, an alloy, a composite, an organic material, a cyborg, or a combination thereof. The target substrate may be of a solid, a liquid state or a transitory phase state.

The method may be regarded as an electrostatic-assisted fully dry transfer technique. When used with chemical vapour deposition to deposit the at least one carbon layer on the growth substrate, as compared to conventional wet transfer methods, the use of chemical vapour deposition and electrostatic-assisted fully dry transfer technique in the method may result in the carbon layers having minimal or no metal residue, solvent residue, polymer residue or chemical residues.

In an example, the method may comprise the steps of (I) growing a carbon layer on a growth substrate; (II) applying a ferroelectric polymer layer on the carbon layer and thereby sandwiching the carbon layer between the ferroelectric polymer layer and the growth substrate (III) polarizing the applied ferroelectric polymer layer; (IV) applying a release tape onto the ferroelectric polarized polymer layer and thereby sandwiching the ferroelectric polymer layer between the thermal release tape and carbon layer; (V) roller-assisted mechanical peeling of the carbon/ferroelectric polymer layers from the growth substrate at a peeling speed in excess of or at least about 101 mm/s (if cracks are desired in this step) or at a peeling speed at or below about 100 mm/s (if cracks are not desired at this step) and optionally; (VI) applying a pressure in excess of or at least about 0.5 N/cm² on the ferroelectric polymer layer before performing step (V), while performing step (V), or after performing step (V), to form a plurality of cracks in the carbon layer along a first directional axis, the cracks being spaced apart from each other in a periodic manner along a second directional axis, wherein the second directional axis is substantially perpendicular to the first directional axis in the same plane of the carbon layer; and (VII) removing the release tape from the ferroelectric polymer layer.

Exemplary, non-limiting embodiments of a bandage comprising a multilayer composite will now be disclosed.

The bandage comprises a multilayer composite, where the multilayer composite comprises at least a carbon layer having a plurality of cracks along a first directional axis, the cracks being spaced apart from each other in a periodic manner along a second directional axis, wherein the second directional axis is substantially perpendicular to the first directional axis in the same plane; and a ferroelectric polymer layer.

The bandage may comprise the multilayer composite disposed on a base substrate. The base substrate may be gauze, silicon, polyethylene terephthalate, polyurethane, paper, glass or a commercially available bandage. The base substrate may comprise an adhesive material to enable adhesion of the bandage onto a body surface of a subject.

The bandage may further comprise at least one electrode capable of being engaged with at least one point within or on a surface of the multilayer composite. The at least one electrode, in use, may be connected to an external electronic device such as an electrical power source, an electronic measurement instrument, a wireless transponder device, a wireless transmitter device or combinations thereof.

The at least two electrodes may be aligned parallel to the line crack direction, that is, along the first directional axis, in order to fully utilise the conductivity of the carbon layer.

The bandage may exhibit antibacterial properties towards gram-positive bacteria such as S. epidermis, S. aureus, S. pyogenes, S. saprophyticus, E. faecalis, S. pneumoniae or combinations thereof.

The bandage may exhibit antibacterial properties towards gram-positive bacteria such as E. coli, Salmonella, Shigella, Enterobacteriaceae, P. aeruginosa, C. trachomatis, Y. pestis, P. mirabilis, E. cloacae, S. marcescens or combinations thereof.

The bandage may have a wound facing surface. The wound facing surface may be hydrophobic and has a water contact angle of more than about 90 degrees, more than about 95 degrees, more than about 100 degrees, more than about 105 degrees, more than about 110 degrees, more than about 115 degrees, more than about 120 degrees, more than about 125 degrees, more than about 130 degrees, more than about 135 degrees, more than about 140 degrees, more than about 145 degrees, or more than about 150 degrees.

Due to the hydrophobic nature of the bandage, the bandage may aid in reducing bleeding and expediting the coagulation process. A dense layer of blood cells and platelets with increased blood wettability can be rapidly formed on the bandage, thus promoting clotting and coagulation. Alternatively, the hydrophobic bandage may act as an impervious layer to prevent blood loss through the bandage (or when used with a dressing or gauze, preventing blood loss through the dressing or gauze as well). The bandage may be able to simultaneously achieve fast clotting with no blood loss, have anti-bacterial property, and clot self-detachment. The non-wetting and blood-repelling features of the bandage may aid in withstanding substantial blood pressure and helping to reduce blood loss and bacteria attachment. The bandage exhibits minimal contact between the clot and the bandage, leading to natural clot detachment after clot maturation and shrinkage, which reduces the peeling tension required to peel off the bandage by about one to two orders of magnitude as compared to a non-hydrophobic bandage. The repellence against water and blood is critical and essential so as to allow the bandage to be easily removed from the wound, once healed, without damage to the fragilely covered wound-beds.

The bandage as disclosed herein is flexible and stretchable.

In the bandage as disclosed herein, the carbon layer may possess a positive charge, and the positive charge is between 5 μC/cm² and 10 μC/cm², 6 μC/cm² and 10 μC/cm², 7 μC/cm² and 10 μC/cm², 8 μC/cm² and 10 μC/cm², 9 μC/cm² and 10 μC/cm², 5 μC/cm² and 6 μC/cm², 5 μC/cm² and 7 μC/cm², 5 μC/cm² and 8 μC/cm², or 5 μC/cm² and 8 μC/cm².

Where positively charged bandage may be able to inhibit bacterial growth and adhesion by disrupting the bacterial cell membranes via electrostatic interaction.

In the carbon layer of the bandage, the carbon of the carbon layer may be attached with a biomaterial. The biomaterial may be bonded with the carbon. The carbon may be modified to enable attachment or bonding with the biomaterial. The carbon may be functionalised with the biomaterial. The biomaterial may be a therapeutic agent or a detection agent.

The therapeutic agent may be an agent capable of exerting a therapeutic effect on a subject, when the multilayer composite is placed on a body surface of the subject. The therapeutic agent may be able to stimulate inflammation, angiogenesis, wound contraction, and remodelling for accelerative wound healing, such as for chronic wounds, bedsore wounds, skin cutting wounds, burn wounds, venous ulcers, arterial ulcers or diabetic (neuropathic foot ulcers).

The therapeutic agent may be an anti-cancer drug, an anti-clotting agent, a wound healing agent, an anti-inflammation agent, an antioxidant, a vitamin or a pain reducing agent. The therapeutic agent is not particularly limited and an exemplary therapeutic agent may include diclofenac, salicylic acid, sulphamethoxypyridazine, phenoxymethylpenicillin, phenol red, valproate, bezafibrate, furosemide, indomethacin, mefenamic acid, piroxicam, tolbutamide, warfarin, cystine, sodium cromoglycate, tetrachlorodecaoxide, hydrogen peroxide, carbamide peroxide, ferric subsulfate, sodium perborate, potassium nitrate, CHS-828, OXI-4503, PX-12, CPI-613, double stranded DNA, single stranded DNA, double stranded RNA, single stranded RNA, messenger RNA or combinations thereof.

The detection agent may act as a probe for a target analyte and be capable of detecting the target analyte, such as when the multilayer composite is placed on a body surface of a mammal; or when made into or as part of a detection device. The detection agent may be an antibody, a protein, a nucleic acid or a combination thereof. The antibody or protein is not particularly limited and an exemplary antibody or protein may include an IgG antibody, an IgA antibody, an IgM antibody, an IgE antibody, an IgD antibody, a SAR-COV 2 antibody, a MERS-COV antibody, a Zika virus antibody, a HIV antibody, a Polio antibody, a Tenanus antibody, an Influenza antibody, an antinuclear antibody, an anti-transglutaminase antibody, an anti-ganglioside antibody, an anti-actin antibody, an anti-thyroid antibody, a spike glycoprotein, a spike protein, a membrane protein, an envelope protein polysaccharide, a peptides, an antigen or a combination thereof.

The carbon of the bandage may be attached with a non-organic material. The non-organic material may be bonded with the carbon. The carbon may be modified to enable attachment or bonding with the non-organic material. The non-organic material may be a nanometal, a nano composite or a nanoalloy.

The nanometal may be a metal that exhibits antibacterial, antiviral, or antifungal properties. The nanometal is not particularly limited and exemplary nanometals may include titanium, cobalt, nickel, copper, zinc, zirconium, molybdenum, tin, lead, silver, or combinations thereof.

The nano composite may be a composite comprising one or more nanometals that exhibits antibacterial, antiviral, or antifungal properties in a matrix. The nanometal is not particularly limited and exemplary nanometals may include titanium, cobalt, nickel, copper, zinc, zirconium, molybdenum, tin, lead, silver, or combinations thereof. The composite matrix may be a metal, a wood, a wood derivative, a polymer, a monomer, a plastic, a ceramic or combinations thereof.

The nanoalloy may be an alloy comprising at least one metal component that exhibits antibacterial, antiviral, or antifungal properties. The nanoalloy components are not particularly limited and exemplary nanometals may include titanium, cobalt, nickel, copper, zinc, zirconium, molybdenum, tin, lead, iron, platinum, silver, aluminium, palladium, gold or combinations thereof.

The biomaterial or non-organic material may be capable of being released from the bandage upon application of an external stimulation to the bandage. The release may be in a controlled manner. The external stimulation may be an electrical stimulation, a thermal stimulation, a physical stimulation, a chemical stimulation, a radioactive stimulation, or a photonic stimulation or combinations thereof. Therefore, where the biomaterial is a therapeutic agent (such as a drug), the bandage may be used to for drug delivery, drug targeting and controllable drug release in order to promote the wound healing process.

The bandage (with or without the biomaterial or non-organic material) may be subjected to an external stimulation, such as those described above, such that when placed on a wound on a subject, the externally stimulated bandage may be capable of attracting stem cells from affected organs of the subject and promote adhesion of the stem cells on the bandage. Following which, the skin regenerative inducers' preconcentration effect dominates and can significantly accelerate the specific differentiation of the stem cells into skin cells. In this way, the bandage can serve as an antibacterial and electroactive platform, capable of simulating cell immigration and proliferation during the wound healing process. Where the bandage contains the biomaterial as mentioned above, this aids in enhancing the therapeutic effect of the bandage, especially with regard to wound healing.

Where the external stimulation is electrical stimulation, electrical stimulation may be carried out by subjecting the bandage to an electrical field. This may be done by connecting an electrode (of positive or negative polarity) to the bandage when placed on the wound on the subject and another electrode placed nearby on intact dry skin of the subject. The pulse frequency and voltage are then set as desired depending on the requirement. As an example, the pulse frequency may be 100 pulses/second and the voltage may be between about 50 to about 150 volts. Depending on the voltage, this delivers a current that is capable of producing a moderately strong but comfortable tingling sensation (insensate skin) or a just-visible muscle contraction (in insensate skin, as in patients with spinal cord injuries).

The bandage may be polarised as desired to address the clinical needs during wound healing. As an example, positive polarity may be used to promote autolysis by attracting negatively charged neutrophils and macrophages, while negative polarity may be used to encourage granulation tissue development by attracting positively charged fibroblasts. As another example, positive polarity may be used to stimulate wound resurfacing by attracting negatively charged epidermal cells.

The constant electric field on the top of the carbon layer, induced by the ferroelectric polymer layer, is also capable of promoting wound healing. The flexible, light, and highly conductive bandage is thus capable of being used together with electrical stimulation devices for accelerative wound healing process.

Exemplary, non-limiting embodiments of a biosensing device comprising a multilayer composite will now be disclosed.

The biosensing device comprises a multilayer composite, where the multilayer composite comprises at least a carbon layer having a plurality of cracks along a first directional axis, the cracks being spaced apart from each other in a periodic manner along a second directional axis, wherein the second directional axis is substantially perpendicular to the first directional axis in the same plane; and a ferroelectric polymer layer.

The biosensing device may be an electrical biosensor. When used in the electrical biosensor, the self-capacitance of the ferroelectric polymer layer of the multilayer composite and electrical resistance of the carbon layer of the multilayer composite can be utilized as two different signalling pathways for real-time monitoring of wound beds. For example, the carbon layer can be utilized as an electrode for the ferroelectric polymer layer, the capacitance value is increased with applied pressure on the multilayer composite. The increase of capacitance/voltage is dependent on the pressure within a specific range (0-300 mmHg), with a rapid recovery time of for example, 0.2 second. At the same time, the electrical resistance of the carbon layer can be significantly decreased (for example, to 200 Ω/sq) in the presence of the ferroelectric polymer layer, thereby improving the detective sensitivity and lowering the limit of detection. The resistance of the carbon layer can be increased by bending the multilayer composite. Due to the combination of the ferroelectric polymer layer and the highly conductive carbon layer, this synergistically enhances the biosensing performance of the biosensor via a multi-channel strategy.

The biosensing device may be an optical biosensor. Here, the ferroelectric polymer layer may comprise a fluorescent agent. The fluorescent agent may be fluorescent nanoparticles, a fluorescent bioprobe or fluorescent quantum dots, which would be readily known to a person skilled in the art. The fluorescent agent may change colour in response to changes in the applied pressure, the surrounding temperature, pH value or other parameters, which can be easily measured by using a fluorescent measuring device or an optical reader.

The biosensing device may be an electrical and optical biosensor, where in the electrical biosensor as mentioned above, the ferroelectric polymer layer used is one that contains the fluorescent agent.

The biosensing device may be a field-effect transistor whereby in the multilayer composite, the carbon of the carbon layer is attached with a detection agent, as mentioned above. Here, the biosensing device can be used to probe for the presence of the respective target analyte in a sample rapidly and with high sensitivity.

Depending on the application of the multilayer composite, whether as part of a bandage of biosensing device, the thickness and size of the multilayer composite may be altered as required in order to change the physical properties of the multilayer composite, allowing the multilayer composite to be tuned to the needs of various applications in therapeutics and biomedicine research as well as their related industries.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 is a schematic diagram of the optical transmission characterization of a multilayer composite as disclosed herein where the carbon layer is graphene within the light spectrum of 350 nm to 800 nm wavelength.

FIG. 2 is a fluorescent microscopy image of bacteria S. epidermidis on a PET substrate with a thin film of the multilayer composite as disclosed herein where the carbon layer is graphene and a ferroelectric polymer.

FIG. 3 is an optical image of the water contact angle profile characterization of the multilayer composite thin film of FIG. 2.

FIG. 4 is a schematic illustration of a bandage of the present disclosure.

FIG. 5a is a schematic illustration of an embodiment of a circuitry to enable biosensing capabilities of a smart bandage embedded with the multilayer composite.

FIG. 5b is a schematic illustration of another embodiment of a circuitry to enable biosensing capabilities of a smart bandage embedded with the multilayer composite.

FIG. 6a is an optical microscopy image of a graphene sheet produced by the method as disclosed herein with a 10 μm scale bar for reference. An axis on the left is used to depict the first directional axis and second directional axis of the periodic cracks on the graphene sheet.

FIG. 6b is an optical microscopy image of a graphene sheet produced by the method as disclosed herein with a 10 μm scale bar for reference. An axis on the left is used to depict the first directional axis and second directional axis of the periodic cracks on the graphene sheet.

FIG. 7 is a graph representing the number of survival bacterial colonies after incubation with different samples to P. aeruginosa NUH9/98 strain.

### EXAMPLES

Non-limiting examples of the invention will be further described in greater detail by reference to specific Examples, which should not be construed as in any way limiting the scope of the invention.

### Example 1—Dry Transfer Technique

The dry phase transfer technique provided by the method as disclosed herein provides for a contamination free graphene manufacture and transfer application. The multilayer composite produced in this manner is easily transferred to universal substrates. Some target substrates that the multilayer composite can be transferred to includes materials such as metals, woods, polymers, ceramics, alloys, and composites.

A PVDF/Graphene thin film produced by the method as disclosed herein was transferred to a variety of solid and soft materials, including silicon disc and wafers, polyurethane, polyethylene terephthalate (PET), paper, and commercially available bandages. In comparison to traditional wet transfer techniques for CVD graphene, the dry phase transfer technique in the method as disclosed herein is completed in totally dry conditions at room temperature, that significantly extends the application range and lifts the restriction on substrates.

In one example, a PVDF-TrFE/graphene multilayer composite is prepared. Graphene is grown on a copper growth substrate via CVD and is then coated with a solution of the ferroelectric polymer using spin-coating method.

PVDF may be dissolved in dimethyl formamide (DMF) to form a solution to be subsequently coated on the graphene. A 500 nm thick film of PVDF can be formed on top of the graphene layer using this method.

After the coating, the film may be annealed to evaporate the solvent and to promote the formation of the ferroelectric phase. The 500 nm thick layer of PVDF film may be annealed at 135° C. between 1 minute and 24 hours.

The resulting thin film polymer layer may be between 300 nm and 2000 nm thick.

After annealing, the dipoles in the ferroelectric polymer film may be aligned perpendicular to the graphene by applying an electric field across the film.

The field can be applied using external electrodes to apply a voltage across the film to ionize the surface of the polymer using voltage of between about 1 kV/cm and about 10 kV/cm. In a PVDF film around 500 nm thick, the dipoles may be aligned by ionizing the surface of the polymer at a voltage of 6 kV/cm.

In some examples, annealing and polarization may be done in a single process. In another version of this step, polarizing the ferroelectric polymer may include applying an external electric field to the polymer layer, such as an external electric field with an electric field strength of between about 50 V/μm and about 500 V/μm. In the case of PVDF as the ferroelectric polymer, a field on the order of 100 V/μm may be required to align the dipoles.

The polarized ferroelectric polymer layer may comprise a remanent polarization of between about 5 μC/cm² and about 10 μC/cm².

Peeling of graphene/ferroelectric polymer from the growth substrate can be completed by applying a peeling force perpendicular to the growth substrate.

After peeling, the PVDF-TrFE/graphene multilayer composite could be incorporated or laminated onto any target substrate as disclosed herein, including another multilayer composite or stack of the same.

### Example 2—Optical Transmission of Multilayer Composite

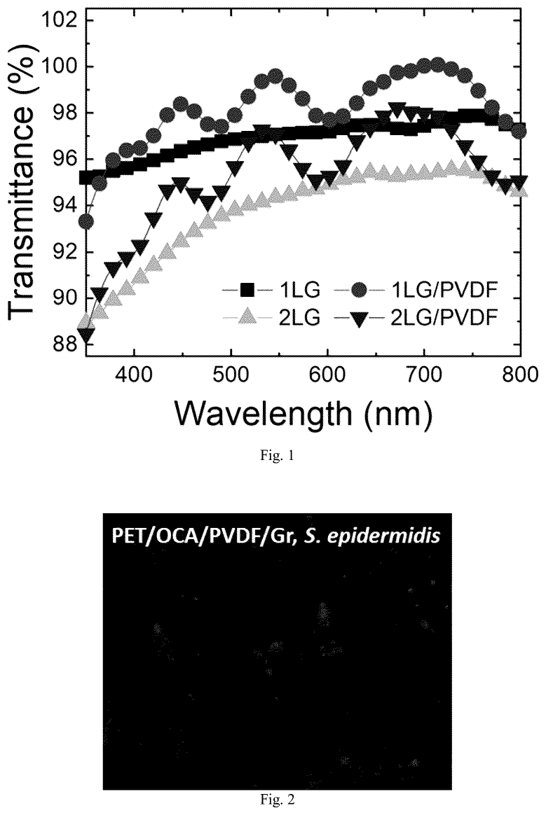

Multiple examples of the carbon layers, ferroelectric polymer layers, and multilayer composite of the present disclosure manufactured by the method as disclosed herein are characterized for their optical transmission. In an experiment, (A) a single layer of graphene, (B) a bilayer graphene, (C) a multilayer composite consisting of a single layer of PVDF-TrFE and a single layer of graphene, and (D) a multilayer composite consisting of a single layer of PVDF-TrFE and a bilayer graphene are characterized across the visible spectrum from 350 nm to 800 nm wavelength (FIG. 1).

When characterized at 550 nm wavelength, (A) the single layer of graphene has an optical transmission of 97.0%, (B) the bilayer graphene has an optical transmission of 94.4%, (C) the multilayer composite consisting of a single layer of PVDF-TrFE and a single layer of graphene has an optical transmission of 99.5% and (D) the multilayer composite consisting of a single layer of PVDF-TrFE and a bilayer graphene has an optical transmission of 97.0%. These high optical transmission values of the graphene and multilayer composites mean they have high transparency and are effective for visual monitoring of wounds when used as a medical bandage.

The (C) multilayer composite consisting of a single layer of PVDF-TrFE and a single layer of graphene also exhibits improved an optical transmission of 99.5% over (A) a single layer of graphene 97.0%. The (D) multilayer composite consisting of a single layer of PVDF-TrFE and a bilayer graphene also exhibits an improved optical transmission of 97.0% over a (B) bilayer graphene 94.4%. This is indicative that the use of the ferroelectric polymer layer in combination with the graphene layer is advantageous as it has synergistic effect of improving the optical transmission.

### Example 3—Antimicrobial p-Doped Graphene Layer

A multilayer composite of the present disclosure, manufactured by the method as disclosed herein using PVDF-TrfE as the ferroelectric polymer layer and graphene as the carbon layer, demonstrates antimicrobial properties. The multilayer composite is polarized and highly charged due to the ferroelectric PVDF-TrFE layer, with the graphene exhibiting a positive charge due to p-doping by the PVDF-TrFE polymer layer. A PET/OCA substrate is prepared by manually aligned lamination of an OCA layer onto a PET substrate. The multilayer composite was placed on the PET/OCA substrate with the PVDF-TrFE polymer layer placed downwards in contact with the PET/OCA surface, and the positively charged graphene layer facing upwards and exposed. Fluorescent-based antimicrobial assay was then conducted on the multilayer composite, with 107 cells per mL of S. epidermidis (ATCC 36984, American Type Culture Collection, Manassas, VA,) exposed to the multilayer composite. Fluorescent microscopy images (FIG. 2) indicates that S. epidermidis were eradicated after 4 hours of exposure to the multilayer composite, and 60% of the S. epidermidis bacteria were eradicated after 6 hours of exposure to the multilayer composite, thus indicating that the multilayer composite exhibits antimicrobial properties. Finally, the infective rate during diverse clinical settings and treatments was effectively reduced, which can aid in improving people's life and the living quality of patients.

In an example with P. aeruginosa, four different film samples were prepared, namely PET, PET/Graphene, PET/PVDF and PET/PVDF/Graphene. Lawn of P. aeruginosa from streak plate were resuspended in 1 mL of PBS to OD600 of 0.5. The initial population densities of P. aeruginosa for experimental samples were maintained at about 10⁸ CFU/mL. Before bacterial incubation, these samples were irradiated for 30 minutes. Next, 20 μL of bacterial suspension (10⁸ CFU/mL) was aseptically transferred onto the 1×1 cm² sample as 10 μL droplets.

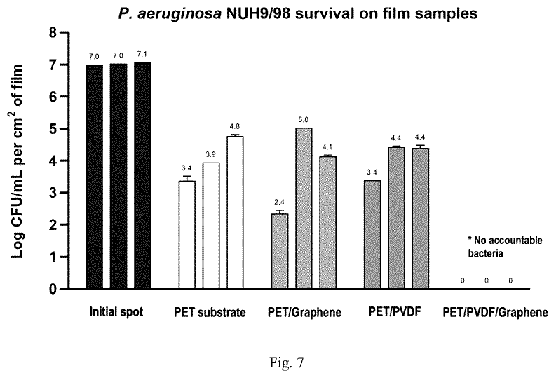

Then, the experimental samples were incubated at 37° C. for 20 hours with aeration. After the experimental treatments, the sample surface was immersed into 1 mL of PBS, followed by 30 seconds of vigorous vortex treatment for complete detachment of adhered bacteria on sample surface. After that, the collected solution was serially diluted 10-fold with PBS and spread plates on LB agar were performed in duplicates. After incubation of plates at 37° C. overnight, the final bacterial colonies were count and recorded (FIG. 7). This assay for each sample was done in triplicates. The 4-log reduction observed after 20 hours of incubation on the PET/PVDF/Graphene film sample surface as compared to the other control samples, indicates that 99.99% of P. aeruginosa are eradicated by the PVDF/Graphene film due to its antibacterial properties.

Due to the high optical transmission of the multilayer composite, a bandage manufactured from the multilayer composite can be highly transparent and antimicrobial, allowing for direct visualization and signalization of wounds without removing the bandage. The changing frequency of a bandage made from the multilayer composite can be about 6 days, which is 6 times longer than that of a conventional bandage.

### Example 4—Hydrophobicity of Graphene Layer

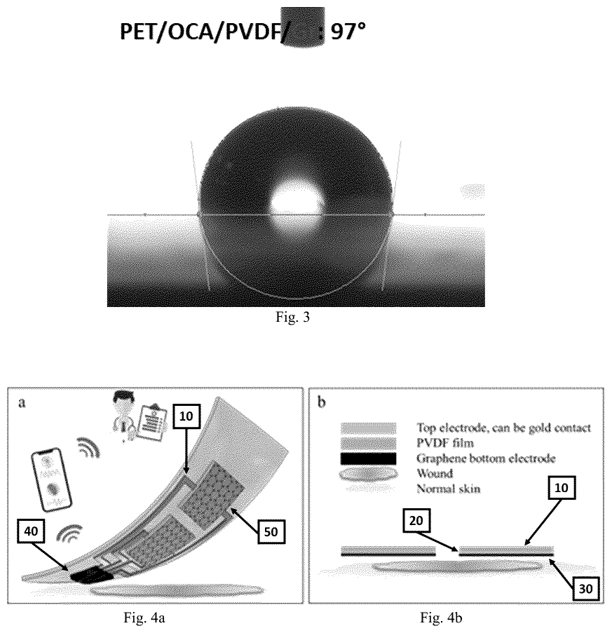

A multilayer composite of the present disclosure, manufactured by the method as disclosed herein using PVDF-TrFE as the ferroelectric polymer layer and graphene as the carbon layer, was demonstrated to be hydrophobic. The multilayer composite was placed on a PET/OCA surface, with the PVDF-TrFE polymer layer placed downwards in contact with the PET/OCA surface, and the graphene layer facing upwards and exposed. A water contact angle characterization test was then conducted by dropping a water droplet onto the exposed graphene surface. The water contact angle was then captured and characterized using a high-resolution optical measurement device after the water droplet had stabilized. The water contact angle profile of the exposed graphene layer was determined to be 97 degrees (FIG. 3), indicating the hydrophobic nature of the graphene layer of the multilayer composite.

### Example 5—Biosensing Modification of Multilayer Composite

With the good flexibility and excellent conductivity of the multilayer composite, a multi-sensing wearable biosensing device is another applicable aspect. A smart bandage is highly desirable for wound management as it offers accurate and real-time monitoring of wound conditions by detecting the relative parameters and signals, such as temperature, pH value, pressure, swelling tension, pus discharge of wound beds.

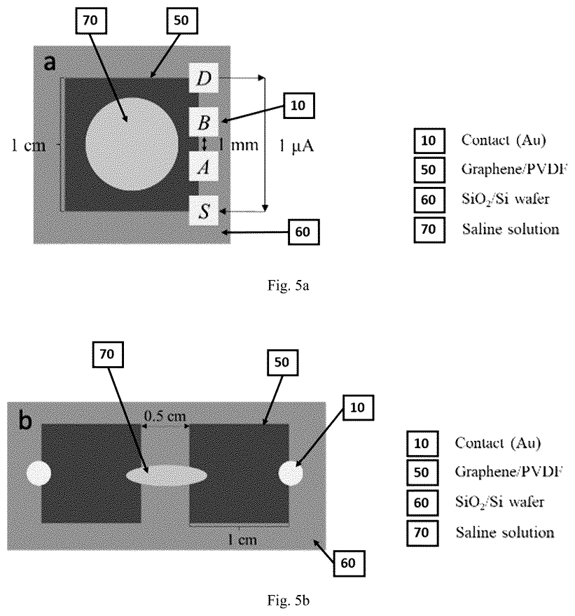

The multilayer composite multilayer composite of the present disclosure manufactured by the method as disclosed herein may be used as a wearable smart bandage with biosensing and wound management capabilities. The multilayer composite may be embedded with a circuitry, similar but not limited to FIG. 5a and FIG. 5b, in the layout similar but not limited to FIG. 4a and FIG. 4b, for multichannel biosensing by the detection of capacitance, resistance, and binary on-off circuitry changes and fluctuations.

In FIG. 4a and FIG. 4b, there is a visualization of an example of a biosensing smart bandage where an electrode 10 can be embedded into a bandage comprising of the multilayer composite 50. Two pieces of pads made from the multilayer composite 50 comprising graphene layer 30 and ferroelectric polymer layer 20 was connected via a gold electrode contact 10 to an electronic device 40. Electronic device 40 may be a transponder or transmitter which may relay information wirelessly or via electrical signals to a medical provider.

In FIG. 5a, there is a visualization of an example of a circuitry to enable biosensing capabilities of a smart bandage embedded with the multilayer composite 50. Electrode 10 contacts can be spaced intermittently across the multilayer composite 50 and detect minute changes in capacitance and resistance across the multilayer composite 50 caused by saline solution 70. Minor variations in the surface temperature and elongation of the smart bandage can be detected by transposing the fluctuations in capacitance and resistance. These parameters may be used to provide information about biometrics such as body temperature, and wound swelling, thus providing biosensing capabilities.

In FIG. 5b, there is yet another visualization of an example of a circuitry to enable biosensing capabilities of a smart bandage embedded with the multilayer composite 50. Electrode 10 contacts can be spaced at opposite ends of two non-contacting multilayer composite 50. When biofluids such as pus, simulated by saline solution 70, are present across the two non-contacting multilayer composite 50, the circuitry will register a drastic drop in open circuit resistance, thus providing information about biometrics such as pus discharge, monitoring of blood coagulation and blood discharge, thus providing biosensing capabilities.

The graphene layer may be utilized as an electrode for a piezoelectric PVDF-TrFE film, wherein the capacitance value was increased with applied pressure on the film. The increase of capacitance/voltage was dependent on the pressure within a specific range (0-300 mmHg), with a rapid recovery time of 0.2 second. At the same time, the electrical resistance of a graphene layer can be significantly decreased to about 200 Ω/sq in the presence of a PVDF-TrFE layer, improving the detective sensitivity and lowering the limit of detection. The resistance of graphene film was increased while bending the PVDF-TrFE/Graphene film, allowing for biosensing of physical changes, such as pus discharge and inflammation swelling.

Non-limiting examples of the multichannel biosensing are as follows:
1) Changes in the wound temperature may be detected by the capacitance changes across the multilayer composite.
2) Swelling of the wound may be detected by the changes in resistance as the multilayer composite elongates in tension due to said swelling.
3) Pus discharge in the wound may be detected by a significant drop in resistance across 2 non-contacting electrodes comprising the multilayer composite.
4) Integration of quantum fluorescent dots into the multilayer composite to obtain a dynamic fluorescent and optical readout response to detect changing pH in the surrounding wound environment.

### Example 6—Strain Induced Periodic Cracks on Carbon Layer

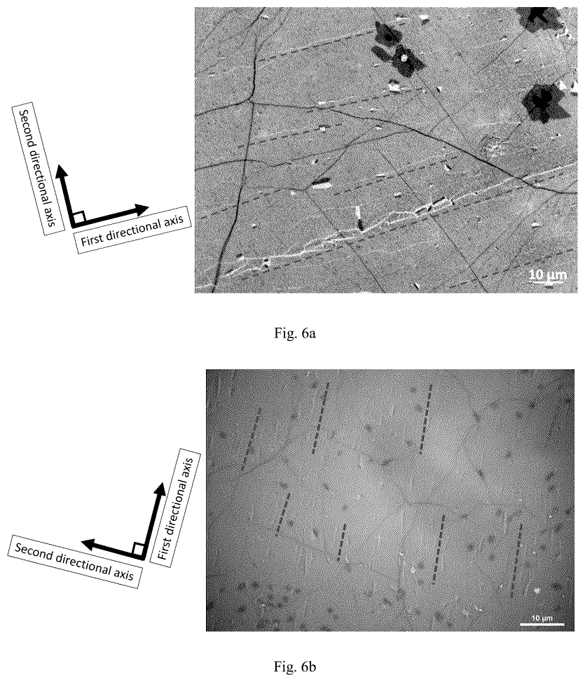

A multilayer composite of the present disclosure manufactured by the method as disclosed herein contains periodic cracks on the surface of carbon layers that are induced by excessive strain applied during the laminating process. Graphene sheets were produced using the method as disclosed herein and characterized using optical image microscopy. The optical images of the graphene sheets (FIG. 6a and FIG. 6b) show high uniformity and coverage with visible one-dimensional periodic cracks (represented by the dotted lines in FIG. 6a and FIG. 6b) in the graphene sheet. The lines that are not indicated with the dotted lines on the optical images of the graphene sheet are wrinkles on the graphene where a portion of the graphene layer has folded in on itself and are not periodic cracks induced by excessive strain (or pressure) or removing the graphene layer from the growth substrate at an increased peeling speed. As seen in FIG. 6a and FIG. 6b, the periodic cracks are along the first directional axis, with the cracks being spaced apart from each other in a periodic manner along the second directional axis, and the second directional axis is substantially perpendicular to the first directional axis in the same plane. These periodic cracks may advantageously improve control of the permeability and breathability of the multilayer composite, thus enhancing the effectiveness of the multilayer composite as a medical bandage.

### Example 7—Promotion of Wound Healing

A multilayer composite manufactured using the method disclosed herein with PVDF-TrFE as the ferroelectric polymer and graphene as the carbon layer serves as an antibacterial and electroactive platform, simulating cell immigration and proliferation during the wound healing process to stimulate inflammation, angiogenesis, wound contraction and remodelling for an accelerative wound healing, especially for chronic wounds and bedsore wounds. The direct contact of graphene onto wounds is capable to promote the related cell proliferation and differentiation, hence accelerating wound healing.

The pristine graphene film is ascertained to control and promote human mesenchymal stem cells' osteogenic differentiation, while the piezoelectric PVDF-TrFE film is reported to enhance cell attachment and blood vessel formation and simulate an electric field for stem cell immigration and proliferation. As for stem cell-based wound healing, it is highly effective and efficient to attract adult multipotent stem cells from local tissue rather than introducing stem cells, for subsequent attachment, proliferation, and specific differentiation.

The simulated electric field from the highly doped PVDF-TrFE/graphene film attracted stem cells from affected human organs and promote its adhesion on the bandage. After that, skin regenerative inducers' pre-concentration effect was dominant, significantly accelerating the specific differentiation into skin cells. Therefore, the multilayer was highly expected to accelerate wound healing and be adapted for different types of wounds, including but not limited to skin cutting wounds, burn wounds, venous ulcers, arterial ulcers and diabetic (neuropathic foot ulcers).

### Example 8—Stimulation Device

A multilayer composite comprising of PVDF-TrFE as the ferroelectric polymer layer and graphene as the carbon layer is conductive, with a sheet resistance of about 200 Ω/sq and is capable of electrical and electronic stimulation. The PVDF-TrFE/Graphene film can also be polarized, with the strength and polarity of the surface charges tailored to fulfil different needs scenarios. For the use of the multilayer composite as a wound bandage, the PVDF-TrFE/graphene film can promote autolysis by using positive charges on the multilayer composite to attract negatively charged neutrophils and macrophages. To encourage granulation tissue development, negative charges on the multilayer composite can be used to attract positively charged fibroblasts. To stimulate wound resurfacing, positive charges on the multilayer composite can be used to attract negatively charged epidermal cells.

Other than using the polarity and surface charge, the conductivity of the multilayer composite also provides for direct electrical stimulation of a wound. Electrical stimulation has been reported to be beneficial for skin wound healing by promoting cell migration and proliferation. Additionally, electrical stimulation could speed wound healing by increasing capillary density and perfusion, improving wound oxygenation, and encouraging granulation and fibroblast activity. In one example, an electrode of either polarity was applied to a sterile, conductive PVDF-TrFE/graphene pad placed on a wound. The conductive surface of the other electrode was applied nearby on intact dry skin. Subsequently, the pulse frequency was set to 100 pulses/second. The voltage was set between 50 and 150 volts to deliver a current that produces a moderately strong but tingling sensation to insensate skin or a just-visible muscle contraction in insensate skin, as in patients with spinal cord injuries.

The conductivity of the multilayer composite can also be exploited for the use of the multilayer composite as a drug delivery platform. Non-covalent functionalization can be employed for surface functionalization of the graphene surface of the PVDF-TrFE/graphene multilayer composite to render solubility, drug-loading capability, and anti-biofouling abilities. In addition, due to the positively charged graphene surface, it can bind, capture, and encapsulate various therapeutics, including anti-cancer drugs, poorly soluble drugs, antibiotics, antibodies, peptides, DNA, RNA and genes.

A PVDF-TrFE/graphene multilayer composite loaded with doxorubicin (DOX) could release drug molecules in response to electrical stimulation, with the amount and release rate controlled by the type and intensity of electrical stimulation applied to the multilayer composite. Thus, with the conductivity and drug loading capabilities of the PVDF-TrFE/Graphene multilayer composite platform, and its ability to serve as both nanocarriers and drug-releasing regulators, the multilayer composite as disclosed herein can serve as a stimulation device for biomedical applications.

### INDUSTRIAL APPLICABILITY

The multilayer composite may be used as a medical bandage with features of transparency for wound monitoring, antibacterial properties to promote wound healing, and sanitization of wounds, and high conductivity to enable smart biosensing through implementation of functional circuitry designs. It may also be used as a therapeutic carrier, electronical stimulator, and cell growth proliferator to enhance wound care and treatment in the medical industry.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1.-23. (canceled)

24. A multilayer composite comprising at least one carbon layer and a ferroelectric polymer layer, wherein the ferroelectric polymer is configured to induce an electric field on one side of the at least one carbon layer.

25. The multilayer composite of claim 24, wherein the at least one carbon layer has a plurality of cracks along a first directional axis, said cracks being spaced apart from each other in a periodic manner along a second directional axis, wherein said second directional axis is substantially perpendicular to said first directional axis in the same plane.

26. The multilayer composite of claim 24, wherein the at least one carbon layer has non-cracks on a surface.

27. The multilayer composite of claim 24, wherein the at least one carbon layer has randomly generated cracks on a surface, and wherein the randomly generated cracks do not form a continuous phase.

28. The multilayer composite of claim 24, wherein said carbon layer is a two-dimensional carbon layer and the carbon of said carbon layer is selected from the group consisting of an amorphous carbon, a graphene, a graphene oxide, a reduced graphene oxide, a graphite or a combination thereof graphene or an amorphous carbon.

29. The multilayer composite of claim 24, wherein said carbon layer has a thickness in the range of 0.34 nm to 100 nm.

30. The multilayer composite of claim 24, wherein the carbon of said carbon layer is attached with a biomaterial or non-organic material, and wherein the ferroelectric polymer of said ferroelectric polymer is selected from the group consisting of fluoropolymers, polyamides, vinyl polymers, copolymers thereof and combinations thereof.

31. The multilayer composite of claim 24, wherein said ferroelectric polymer layer has a thickness in the range of 300 nm to 2000 nm.

32. The multilayer composite of claim 24, wherein the multilayer composite is conductive and exhibits a sheet resistance of between 100 Ω/sq and 200 Ω/sq per bilayer consisting of said carbon layer and said ferroelectric polymer layer.

33. The multilayer composite of claim 24, wherein said multilayer composite comprises a plurality of bilayers, each bilayer consisting of one carbon layer and one ferroelectric polymer layer; or a plurality of stacked layers, each stacked layer consisting of more than one carbon layers and one ferroelectric polymer layer.

34. A method of producing a multilayer composite, comprising the steps of:
(a) providing at least one carbon layer on a growth substrate;
(b) applying a ferroelectric polymer layer on said carbon layer;
(c) polarizing said ferroelectric polymer layer; and
(d) forming a plurality of cracks in said carbon layer along a first directional axis, said cracks being spaced apart from each other in a periodic manner along a second directional axis, wherein said second directional axis is substantially perpendicular to said first directional axis in the same plane of said carbon layer.

35. The method of claim 34, wherein said forming step (d) comprises the step of (d1) applying a pressure in excess of or at least 0.5 N/cm2 on the ferroelectric polymer; or the step of (d2) removing the carbon/ferroelectric polymer layers from the growth substrate at an increased peeling speed; or both steps (d1) and (d2).

36. The method of claim 34, further comprising, before said applying step (d), the steps of:
(d3) applying a release adhesive on said ferroelectric polymer layer; and
(d4) optionally, removing the carbon/ferroelectric polymer layers from the growth substrate.

37. The method of claim 36, further comprising, after said applying step (d) the step of:
(d5) removing said release adhesive from said ferroelectric polymer layer.

38. The method of claim 36, wherein said applying step (d) or applying step (d5) is undertaken at a temperature in the range of 30° C. to 160° C.

39. The method of claim 34, wherein said providing step (a) comprises the steps of:
(a1) applying a first carbon layer on said growth substrate;
(a2) applying a second carbon layer on said first carbon layer; and
(a3) repeating said applying step (a2) for one to three times.

40. The method of claim 34, further comprising the steps of:
(e) repeating steps (a) to (d) to form a subsequent multilayer composite; and
(f) laminating said subsequent multilayer composite onto a multilayer composite previously produced by steps (a) to (d) or steps (a) to (f).

41. The method of claim 34, wherein said polarizing step (c) comprises introducing an external electric field with opposite polarities across both surfaces of said ferroelectric polymer layer, said surfaces being opposite to each other.

42. A bandage for the promotion of wound healing comprising a multilayer composite, said multilayer composite comprising at least one carbon layer and a ferroelectric polymer layer, wherein the ferroelectric polymer is configured to induce an electric field on one side of the at least one carbon layer.

43. The bandage of claim 42, wherein the at least one carbon layer has a plurality of cracks along a first directional axis, said cracks being spaced apart from each other in a periodic manner along a second directional axis, wherein said second directional axis is substantially perpendicular to said first directional axis in the same plane.

44. The bandage of claim 42, wherein the at least one carbon layer has non-cracks on a surface.

45. The bandage of claim 42, further comprising at least one electrode in contact with at least one point within or on a surface of the multilayer composite.

46. The bandage of claim 42, wherein the carbon of said carbon layer is attached with a biomaterial or non-organic material.

47. The bandage of claim 46, wherein said biomaterial or non-organic material is capable of being released from said bandage upon application of an external stimulation to said bandage.

48. The bandage of claim 42, wherein the multilayer composite has antimicrobial properties towards gram-positive bacteria or gram-negative bacteria.

49. A biosensing device comprising a multilayer composite, said multilayer composite comprising at least one carbon layer and a ferroelectric polymer layer, wherein the ferroelectric polymer is configured to induce an electric field on one side of the at least one carbon layer.

50. The biosensing device of claim 49, further comprising at least one electrode in contact with at least one point within or on a surface of the multilayer composite.

51. The biosensing device of claim 49, wherein the electrode is connected to an electronic device.
